(19) United States
(12) Patent Application Publication
(10) Pub. No.: US 2023/0414337 A1
(43) Pub. Date: Dec. 28, 2023

(54) Embolic Protection Device

(71) Applicant: Fortuna Clinical, LLC, Birmingham, AL (US)

(72) Inventors: Jan Skowronski, Birmingham, AL (US); Mark Carlson, Fox River Grove, IL (US)

(73) Assignee: Fortuna Clinical, LLC, Birmingham, AL (US)

(21) Appl. No.: 18/213,178

(22) Filed: Jun. 22, 2023

Related U.S. Application Data
(60) Provisional application No. 63/354,668, filed on Jun. 22, 2022.

(51) Int. Cl. A61F 2/01

(52) U.S. Cl. CPC A61F 2/0105; A61F 2/011

(57) ABSTRACT

The present disclosure includes apparatuses and methods for a distal protection system. The system may include a delivery catheter, an embolic protection apparatus, and a retrieval catheter. An improved embolic protection apparatus which includes a wire, an expandable loop connected to a porous filter, with two legs used to connect the loop to the wire is provided. Embolic protection apparatuses that can be used in a wide diameter range of lumens is disclosed.

### CROSS-REFERENCE TO RELATED APPLICATIONS

This application claims the benefit of U.S. Patent Application No. 63/354,668, filed Jun. 22, 2022, the entire content of which is hereby incorporated herein by reference.

### TECHNICAL FIELD

This application is directed to embolic protection devices, systems, and methods for providing embolic protection.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death for in the United States while PAD is the leading cause of amputation in patients.

Coronary artery disease (CAD) and Peripheral artery disease (PAD) are both caused by the progressive narrowing of the blood vessels most often caused by atherosclerosis, the collection of plaque or a fatty substance along the inner lining of the artery wall. Over time, this substance hardens and thickens, which may interfere with blood circulation to the arms, legs, stomach and kidneys. This narrowing forms a lesion, completely or partially restricting flow through the artery. Blood circulation to the brain and heart may be reduced, increasing the risk for stroke and heart disease.

The procedures used to treat occlusive vascular diseases, such as angioplasty, atherectomy and stent placement, often cause blood clots to form and/or atheromatous material to dislodge from inside the vessel walls and enter the bloodstream. The dislodged material (e.g., plaque), known as at atheroemboli, may be large enough to occlude downstream vessels, potentially blocking blood flow to tissues. Additionally, the blood clots, known as thromboemboli, may be large enough to block the blood flow downstream.

There are numerous previously known interventional systems and methods that employ a filter mechanism designed to capture material dislodged from vessel walls during the treatment or diagnosis of vascular disease. Many of the more recent devices employ an expandable filter disposed at the distal end of a guide wire. These filters have various configurations, such as mesh or microporous membranes in the form of sleeves, parachutes or baskets attached to the guide wire or other delivery mechanism by means of struts, wires, ribs or frames. The meshes are frequently made of woven or braided fibers or wires made of stainless steel, nitinol, platinum alloy, polyester, nylon or porous plastics, for example. The microporous membranes are typically made of a polymer material such as polypropylene, polyurethane, polyester, polyethylene terephthalate, polytetrafluoroethylene or combinations thereof.

As the human body has arteries of various sizes, users of embolic protection devices must stock a variety of sizes as know embolic protection devices are indicated only for a small range of vessel sizes. For example, coronary arteries are generally up to 5 mm in diameter and non-aortic peripheral blood vessels are generally up to 10 mm in diameter. An embolic filter designed to work with a broad range of vessel sizes is described herein.

There remains a need for novel embolic protection devices that are more capable than existing devices, for example being capable of operating under a greater range of conditions and vessel sizes.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF THE DISCLOSURE

Described herein are embolic protection devices deployed in a body vessel or cavity for the collection of loosened and/or dislodged debris.

In one embodiment, the distal protection device comprises a guide wire with an expandable loop and a filter mounted on the wire near the distal end. The expandable loop includes two legs, connected to the expandable loop, that extend at an angle of approximately 90° from the loop and that are connected to the wire.

In another embodiment, the embolic protection device comprises a guide wire with an expandable loop and a filter mounted on the wire near the distal end. The expandable loop includes two legs, connected to the expandable loop, that extend at an angle of approximately 90° from the loop and that are connected to the wire. Positioned 180° from the wire the expandable loop comprises a preferential bend region which may comprise a small u-shaped section of the wire of the expandable loop.

In another embodiment, the embolic protection device comprises a guide wire with an expandable loop and a filter mounted on the wire near the distal end. The expandable loop includes two legs, connected to the expandable loop, that extend at an angle of approximately 90° from the loop and that are connected to the wire. In an at rest configuration, when the expandable loop is not confined in a structure such as a body lumen, the loop has an oval or elliptical shape, with the smaller diameter of the loop being perpendicular to the guide wire.

In another embodiment, the filter material has a plurality of holes. In order to increase the efficiency of the filter, a maximum number of holes is preferred. In this embodiment, the holes are arranged to avoid having a perforation or preferential tear line in the filter.

In another embodiment, the apparatus includes delivery catheter sized to contain an embolic protection apparatus. The delivery catheter may be used to cross lesions. In some examples, the distal end of the wire is positioned outside the distal end of the delivery catheter when crossing a lesion. In some examples, a retrieval catheter is used to capture the expandable distal protection apparatus for removal from the body.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

FIG. 1A is a schematic diagram of an embolic protection device in accordance with a number of embodiments of the present disclosure.

FIG. 1B is a schematic diagram of an embolic protection device in accordance with a number of embodiments of the present disclosure.

FIG. 2 is a schematic diagram of the expandable frame for an embolic protection apparatus in accordance with a number of embodiments of the present disclosure.

FIG. 3 is a schematic diagram of a distal facing view of an embolic protection apparatus in accordance with a number of embodiments of the present disclosure.

FIG. 4 is a schematic diagram of the expandable frame for an embolic protection apparatus in accordance with a number of embodiments of the present disclosure.

FIG. 5 is a schematic diagram of the filter material for an embolic protection apparatus in accordance with a number of embodiments of the present disclosure.

FIGS. 6A and 6B show an embodiment of the invention used in two different diameter blood vessels.

FIGS. 7A and 7B show an embodiment of the invention used in two different diameter blood vessels.

### DETAILED DESCRIPTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the Figures shall refer to like features unless otherwise indicated.

The present disclosure includes methods and apparatuses for devices for providing embolic protection. In one embodiment, the distal protection device comprises a guide wire with an expandable loop and a filter mounted on the wire near the distal end. The expandable loop includes two legs, connected to the expandable loop, that extend at an angle of approximately 90° from the loop and that are connected to the wire. In one embodiment, the expandable loop comprises a preferential bend region which may comprise a small u-shaped section of the wire of the expandable loop. In one embodiment, the expandable loop comprises an oval or elliptical shape, with the smaller diameter of the loop being perpendicular to the guide wire. In one embodiment, the filter material is provided with a plurality of holes with the holes aligned so as to avoid a perforation of preferential tear line. In one embodiment, the distal protection device is designed to work in a wide range of lumen diameters.

An example apparatus includes delivery catheter sized to contain an embolic protection apparatus. The delivery catheter is used to cross lesions. In some examples, the distal end of the wire is positioned outside the distal end of the delivery catheter when crossing the lesion. In some examples, a retrieval catheter is used to capture the expandable distal protection apparatus for removal from the body.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and structural changes may be made without departing from the scope of the present disclosure.

As used herein, designators such as “X”, “Y”, “N”, “M”, etc., particularly with respect to reference numerals in the drawings, indicate that a number of the particular feature so designated can be included. It is also to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of pivot points) can refer to one or more pivot points, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically or for access to and movement of the movable handle member, as appropriate to the context.

As will be appreciated, elements shown in the various embodiments herein can be added, exchanged, and/or eliminated so as to provide a number of additional embodiments of the present disclosure. In addition, the proportion and/or the relative scale of the elements provided in the Figures are intended to illustrate certain embodiments of the present disclosure and should not be taken in a limiting sense.

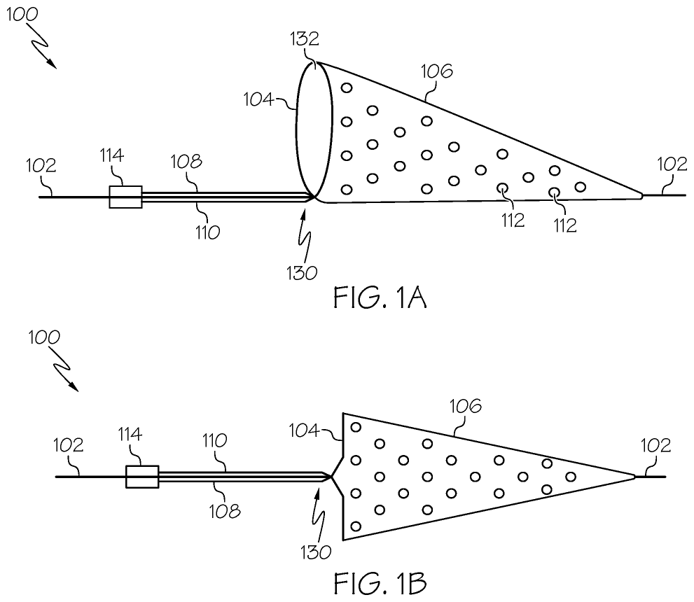

FIG. 1A is a schematic diagram of a perspective view of an embolic protection device in accordance with a number of embodiments of the present disclosure. In the example, embolic protection device 100 can include guide wire 102 that is attached to the expandable frame 104. In some examples, the guide wire extends through the interior of the filter 106 and is attached to the distal end of the filter material 106. Filter 106 comprises a plurality of holes 112. Expandable frame includes legs 108 and 110, attached to the frame at an approximate 90° angle. In some embodiments, this angle is between 70° and 90° or between 80° and 100°. In some embodiments, legs 108 and 110 and frame 104 are a made from a single piece of wire heat set into the proper shape. The proximal ends of legs 108 and 110 are attached to wire 102 at 114, with the legs having a length of about 2 to 20 mm, preferably 5 to 10 mm. Junction 114 can comprise a weld, glue, or other bonding agent or can comprise a sleeve heat sealed, shrunk, or bonded over legs 108 and 110 and to wire 102.

FIG. 1B is a schematic diagram of a bottom view of an embolic protection device in accordance with a number of embodiments of the present disclosure. In the example, embolic protection device 100 can include guide wire 102 that is attached to the expandable frame or loop 104. Expandable frame 104 includes legs 108 and 110, attached to the frame at an approximate right angle. In some embodiments, legs 108 and 110 and frame 104 are a made from a single piece of wire heat set into the proper shape. As shown, leg 110 is positioned on one side of wire 102, extending from junction or connection 114 to expandable loop 104. The other leg is positioned on the other side of wire 102, also extending from junction 114 to expandable loop 104. As shown, the two legs 108 and 110 extend in a proximal direction from frame 104 but in other embodiments they can extend is a distal direction. In some embodiments, the legs 108 and 110, including the approximate right angle as they transition to loop 104, are positioned on a side of the wire opposite of loop 104 such that they cross each other with wire 102 positioned between this crossing and the loop 104.

As shown in FIG. 1B, in some embodiments, one first wire of the two legs crosses the second wire. Following the path of the wire that forms the legs 108 and 110 and frame 104, the wire of first leg 110 starts at junction 114 and extends along a first side of wire 102. Prior to forming frame 104, the first wire crosses the second wire and extends under wire 102 and into the frame 104 on the second side of wire 102. The wire of frame 104 continues from the second side of wire 102, on the top of wire 102, to the wire of frame 104 on the first side of wire 102. The wire of frame 104 then extents under wire 102, crosses the first leg 110 and then forms the second leg 108 extending on the second side of wire 102 until it reaches the junction 114. While this description is for a single piece of wire, multiple pieces of wire, including pieces with different dimensions or physical properties, can be used. While this description has a specific number of crossings, bends and changes of direction, the description here is a minimum; other and/or additional crossings, bends, changes in direction can also be used.

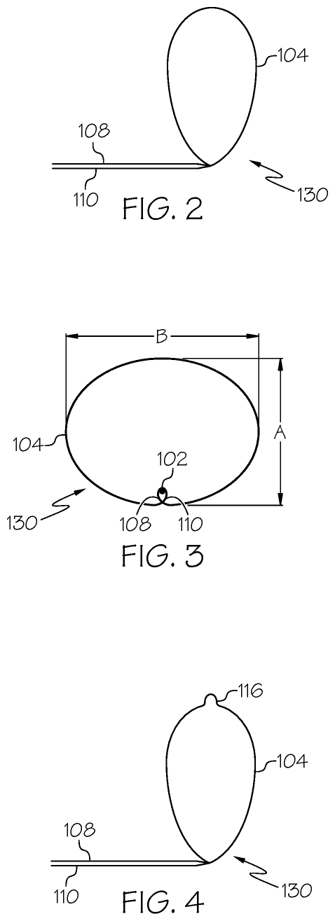

FIG. 2 is a diagram of the wire of legs 108 and 110 and loop 104 in accordance with a number of embodiments of the present disclosure. As shown, the wire of loop 104 begins with leg 108 and extends in a direction, then extends through an approximate right angle, then extends around loop 104, then extends through another approximate right angle and crosses over or under leg 108 before retuning in the direction that leg 108 extended.

FIG. 3 is a view of an embodiment from the proximal end (looking distal) of an embolic protection device in accordance with a number of embodiments of the present disclosure. In this embodiment, loop 104 has an elliptical, oval, egg, or other not-quite round shape when the distal protection device is expanded, at rest, and not constrained in a lumen or other structure. In this embodiment, distance A which extends across loop 104 in a direction perpendicular to and through wire 102 has a vertical distance that is smaller than horizontal distance B which extends across loop 104. In some embodiments, distance B is about 5%, 10%, 15%, 20%, or more than distance A. The not round shape helps ensure that loop 104 is positioned to be in contact with the walls of a lumen when device 100 is placed in a lumen. In some prior art devices, certain parts of an expandable loop may be pulled away and not in contact with a lumen wall when a device is placed in a lumen, especially when placed in tortuous lumens such as blood vessels. In one non-limiting embodiment, to create an embolic protection device that will work in vessels from 4 mm to 10 mm in diameter, distance A is approximately 9.2 mm and distance B is approximately 11 mm.

In some embodiments, an elliptical shape of the loop 104 can be taller than it is wide. In some embodiments, distance A is about 5%, 10%, 15%, 20%, or more than distance B.

FIG. 4 is a schematic diagram of an expandable loop 104 which comprises a preferential bend region 116 in accordance with a number of embodiments of the present disclosure. Preferential bend region 116 may comprise a u-shaped, v-shaped, or omega-shaped section of the wire of the expandable loop 104. Preferential bend region 116 is positioned approximately 180° from legs 108 and 110. Preferential bend region 116 aids in collapsing the distal protection device into the delivery catheter and/or a retrieval catheter as loop 104 will readily bend at this point and easily collapse into the lumen of the delivery and/or retrieval catheter. In one non-limiting embodiment, the wire of loop 104 is constructed of Nitinol with a diameter of 0.006 inches and the preferential bend region bend region 116 has a radius of 0.020 inches and a depth of 0.030 inches to 0.040 inches. A preferential bend region 116 can comprise any suitable shape that will encourage deformation of the loop 104 at the preferential bend region 116. Although FIG. 4 shows a preferential bend region 116 protruding outside of the basic cavity formed by the loop 104, in some embodiments, the preferential bend region 116 can extend into the cavity. In some embodiments, a preferential bend region 116 comprises a higher degree of curvature than other portions of the loop 104. In some embodiments, the preferential bend region 116 comprises one or more inflections in the direction of curvature.

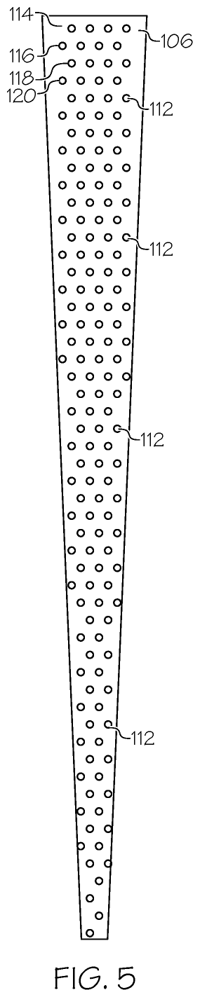

FIG. 5 is a schematic diagram of the filter material 106 in accordance with a number of embodiments of the present disclosure. Holes 112 are formed in filter material 106 so as to maximize the number of holes without appreciably weakening filter 106 by forming a preferential tear or perforation line. A maximum number of holes are desired in filter 106 so as to increase fluid flow when the embolic protection device is in use. For example, to increase blood flow when the filter is used in an artery or vein. Fluid flow could be increased by increasing the diameter of the holes, but this will lead to a less efficient filter as larger particles will go through the filter. As shown in FIG. 5, filter 106 comprises a plurality of holes 112. The strength of the filter material is influenced by the number of holes and the hole center to hole center distance of the holes. In order to increase the number of holes and to keep an acceptable hole center to hole center distance, the holes are staggered along the longitudinal axis direction of the filter. 114, 116, 118, and 120 indicate a row of holes extending around the filter. As shown, the holes in rows 114 and 118 are relatively aligned in the direction of the longitudinal axis of the filter. Likewise, the holes in row 116 and 120 are likewise aligned. The holes in rows 116 and 120 are staggered from the holes in rows 114 and 118 in that the holes in 116 and 120 generally line up with the material between the holes of rows 116 and 120. This staggering pattern limits the formation of a perforation or preferential tear line.

In some embodiments, filter 106 comprises thermoplastic polymers or thermoplastic elastomers. In some embodiments, filter 106 comprises thermoplastic polyurethanes or thermoplastic polyurethane elastomers. In some embodiments, filter 106 comprises polyurethane. In some embodiments, filter 106 comprises thermoplastic polyurethanes including aromatic thermoplastic polyurethanes. In some embodiments, filter is made from a flat piece of polymer that is rolled and sealed in its conical shape. In some embodiments, filter 106 is formed in a conical shape. In some embodiments, the thickness of filter 106 is 0.02 to 0.1 mm or from 0.025 to 0.05 mm. In one embodiment, the thickness of filter 106 is between 0.03 mm and 0.04 mm. In some embodiments, holes 112 can have a diameter of 0.1 to 0.2 mm, or from 0.14 to 0.16 mm. In one embodiment, the diameter of holes 112 is 0.15 mm. In some embodiments, the hole center to hole center spacing of holes 112 is 0.2 to 0.5 mm or from 0.3 to 0.4 mm. In one embodiment, the hole center to hole center spacing of holes 112 is 0.35 mm.

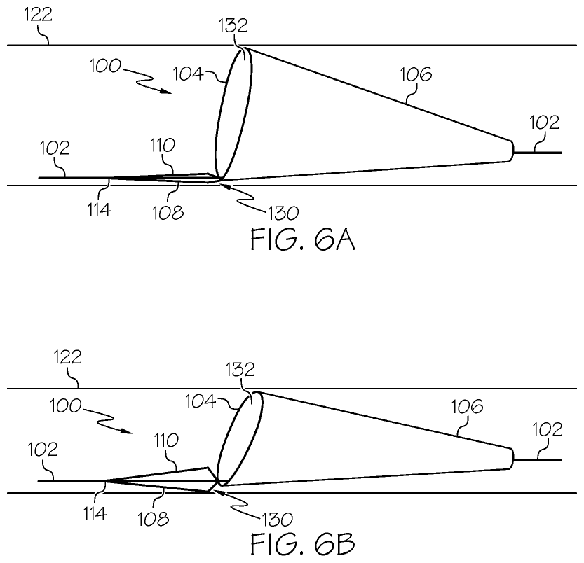

FIGS. 6A and 6B show a distal protection device deployed in lumen 122. In some embodiments, the embolic protection device 100 is designed to work in a large range of lumen diameters. In one example, device 100 will work in lumens ranging from 4 to 12 mm or from 5 to 10 mm. When positioned in larger diameter lumens, legs 110 and 108 are approximately or substantially parallel to wire 102. When positioned in smaller diameter lumens, legs 108 and 110 extend at an angle from wire 102. The proximal ends of legs 108 and 110 are attached to wire 102 at 114. When positioned in a smaller diameter lumen, each of legs 108 and 110 extend from wire 102 at an angle of 5° to 25°. In FIG. 6A, lumen 122 has a large diameter. Loop 104 is positioned approximately perpendicular to lumen 122 and wire 102 and legs 108 and 110 extend approximately parallel to wire 102. In FIG. 6B, lumen 122 has a smaller diameter. Loop 104 is positioned at an angle to lumen 122 and wire 102 and legs 108 and 110 extend at an any away from wire 102 and connection 114. In one embodiment where lumen 122 is a blood vessel, FIG. 6A represents a blood vessel with a diameter from 8 to 12 mm and FIG. 6B represents a blood vessel from 4 to 7 mm.

In some embodiments, an embolic protection device 100 comprises a guide wire 102, a support member 130 and a filter 106. In some embodiments, the support member 130 comprises a first leg 108, a loop 104 and a second leg 110 as described herein. In some embodiments, the support member 130 comprises a single piece of material. In some embodiments, the support member 130 comprises a continuous wire. In some embodiments, the support member 130 is attached at a first end to the guide wire 102 at the junction 114 and extends as the first leg 108 along a length of the guide wire 102, positioned to a first side of the guide wire 102. In some embodiments, the support member 130 then bends and transitions into the loop 104, which can be shaped as described herein and comprises an aperture 132. In some embodiments, the support member 130 crosses itself and bends at the transition from the loop 104 into the second leg 110. The second leg 110 can be located to a second side of the guide wire 102, and the second end of the support member 130 can be attached to the guide wire 102 at the junction 114. In some embodiments, a first portion of the support member 130 crosses over a second portion of the support member 130.

In some embodiments, the first leg 108 is positioned on a first side of a bisecting longitudinal plane and the second leg 110 is positioned on the second side. In some embodiments, the support member 130 crosses the bisecting longitudinal plane at the transition from the first leg 108 into the loop 104. Thus, in some embodiments, the first leg 108 is attached to a portion of the loop 104 oriented on the second side of the bisecting longitudinal plane. In some embodiments, the loop 104 crosses the bisecting longitudinal plane at a location across the aperture 132 from the legs 108, 110. In some embodiments, the support member 130 again crosses the bisecting longitudinal plane at the transition from the loop 104 into the second leg 110. Thus, in some embodiments, the second leg 110 is attached to a portion of the loop 104 oriented on the first side of the bisecting longitudinal plane.

In some embodiments, an end of the filter 106 material is attached to the loop 104 and the aperture 122 comprises an entrance into the filter 106 cavity. In some embodiments, the guide wire 102 passes through the interior of the aperture 122 and extends inside of the filter 106 cavity. In some embodiments, a distal end of the filter 106 is attached to the guide wire 102.

In some embodiments, the support member 120 is attached to the guide wire 102 only at the junction 114. Thus, in some embodiments, the legs 108, 110 are only attached to the guide wire 102 at their proximal ends and the loop 104 is not specifically attached to the guide wire 102. This configuration allows for greater flexibility for the loop 104 to adjust in size in response to smaller vessels, as the loop 104 is able to move away from the guide wire 102. Also, the legs 108, 110 are able to pivot at the junction 114, allowing the distal ends of the legs 108, 110 to move away from the guide wire 102 with the loop 104, and also allowing the distal ends of the legs 108, 110 to spread laterally as the loop 104 is compressed in size.

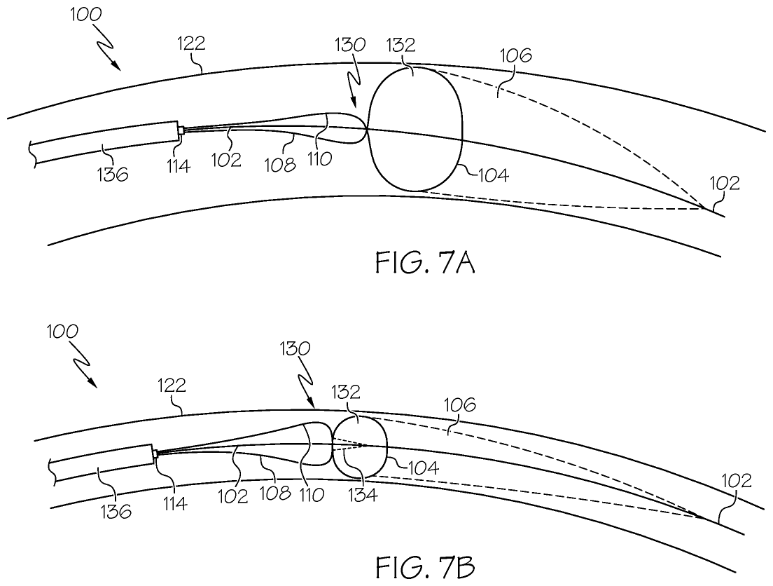

FIGS. 7A and 7B show additional views of an embodiment of an embolic protection device oriented in different sized lumens 122. FIG. 7A shows a larger lumen 122 and FIG. 7B shows a smaller lumen. The loop 104 in FIG. 7B defines a smaller aperture 132, as the size of the loop 104 has been reduced due to compressive forces, for example applied by the lumen 122 wall. As the aperture 132 is reduced in size, a location where the support member 130 crosses over itself can change and the legs 108, 110 can spread laterally, moving away from the guide wire 102. In some embodiments, the filter 106 material can fold and overlap 134 as the aperture 132 is reduced in size. FIGS. 7A and 7B also show an embodiment of a catheter 136.

In some embodiments, an embolic protection device 100 is capable of assuming several different deployed orientations, for example in response to external forces applied by various sized vessels 122. In some embodiments, an embolic protection device 100 comprises a first orientation, for example as shown in FIGS. 6A and 7A, and a second orientation, for example as shown in FIGS. 6B and 7B. In some embodiments, a cross-sectional area of the aperture 132 defined by the loop 104 is larger in the first orientation and smaller in the second orientation. In some embodiments, an angle between the legs 108, 110, for example measured from the junction 114, is greater in the second orientation than in the first orientation. In some embodiments, a spacing between the proximal ends of the legs 108, 110 is greater in the second orientation than in the first orientation. In some embodiments, an angle between a portion of the guide wire 102 located between the legs 108, 110 and the cross-sectional face of the aperture 132 comprises an oblique angle. In some embodiments, the angle between the guide wire 102 and the cross-sectional face of the aperture 132 is greater in the second orientation than in the first orientation. In some embodiments, the loop 104 and legs 108, 110 collectively pivot away from the guide wire 102 at the junction 114 as the support member 130 deforms in response to external forces. Thus, in some embodiments, the distal ends of the legs 108, 110, and a portion of the loop 104, are spaced farther away from the guide wire 102 in the second orientation than in the first orientation. In some embodiments, the filter 106 generally comprises a single layer of material at its connection to the loop 104 in the first orientation. In some embodiments, a portion of the filter 106 can fold over itself and form an overlap 134 in the second orientation. In some embodiments, the overlap 134 is located adjacent to the loop 104. In some embodiments, an overlap 134 is located opposite from the legs 108, 110.

U.S. patent application Ser. No. 17/556,967, published as US2022/0192689, is incorporated herein by reference in its entirety.

In a number of embodiments, some of the sections of wire 102 have different diameters. In some embodiments, the diameter of the proximal section of the wire 102 is of a larger diameter than the wire that extends with filter 106. Many of the commercially available devices for treating peripheral vascular disease are compatible with wires having a diameter of up to 0.035 inches. Many commercially available devices for treating coronary vascular disease are compatible with wires having a diameter up to 0.014 inches. While larger diameter wires provide more support than smaller diameter wires, the smaller diameter wires are generally more flexible and can more easily traverse tortuous anatomy. In some embodiments of the invention, the embolic protection apparatus 100 is positioned within a delivery catheter. For delivery, the combined delivery catheter and embolic protection apparatus 100 are advanced together through a lumen and/or across a lesion. Crossing lesions, especially in tortuous anatomy, requires balancing many features, including pushability and flexibility. Thus, while a smaller diameter device will generally be more flexible, a larger diameter and/or stiffer device will have greater pushability. The diameter of the delivery catheter is dependent on the minimum inside diameter needed to house the collapsed embolic protection apparatus 100 which includes wire 102. In the section of filter that extends from 114 to the distal end of filter 106, the effective diameter will comprise the diameter of the guide wire 102 plus the space needed for legs 108 and 110 and collapsed proximal loop 104 and filter 106. In some embodiments, the competing needs of a large diameter wire needed for support and a small diameter profile needed for the delivery catheter is balanced by using a wire with a diameter over 0.030 inches for the proximal section (proximal of 114) of the guidewire 102 and using a wire with a diameter less than inches for the section of wire 102 extending from 114 to the distal end of filter 106. In other embodiments, the diameter of the section of wire 102 extending from 114 to the distal end of filter 106 is less than 0.025 inches or less than 0.020 inches. In some embodiments, the diameter of the section of wire 102 distal to the distal end of filter 106 will be approximately equal to the diameter of wire proximal to 114. In some embodiments, the diameter of the section of wire 102 distal to the distal end of filter 106 will be between about 0.010 and 0.025 inches. In some embodiments, the section of wire 102 distal to the distal end of filter 106 will comprise three subsections. The proximal subsection will have a wire diameter of greater than 0.30 inches, in some embodiments 0.035 inches. The middle subsection will have a wire diameter between 0.015 and 0.03 inches or between 0.02 and 0.03 inches, is some embodiments 0.18 inches. The distal subsection will have a wire diameter less than 0.02 inches or less than 0.015 inches, in some embodiments 0.014 inches.

In some embodiments the delivery catheter embolic protection apparatus 100 is advanced in a body lumen that may include an occlusion. In some embodiments, the section of wire 102 that extends distal of filter 106 is located distally of the distal end of delivery catheter and is looped back toward the proximal end. This looped back wire may assist the physician/operator to advance the delivery catheter and embolic protection device through the lumen and/or lesion. When located in the proper position, the delivery catheter can be retracted such that expandable loop 104 and porous filter 106 are expanded. Expandable loop 104 expands into contact with the walls of the lumen and porous filter 106 opens. The diameter of expandable loop 104 is sized to be equal to or slightly larger than the diameter of the target lumen so that it touches the inside surface of the lumen. In some examples expandable loop 104 is radiopaque so that the physician/operator can ensure that it has expanded into contact with the wall of the lumen. Expandable loop 104 can be made more radiopaque through the use of platinum, tungsten, or gold markers crimped onto or applied to the loop. After the filter 100 is properly positioned in a lumen, the delivery catheter can be removed from the body. At this point the physician/user may use an interventional device such as a balloon catheter, drug coated balloon catheter, stent delivery catheter, drug coated stent delivery catheter, thrombectomy catheter, IVUS, other imaging catheter, or atherectomy catheter may be used. When a balloon catheter or other interventional device is advanced through the lumen and/or lesion and when it is expanded, or for other interventional devices, used, embolic particles may be released. The embolic particles can be thromboembolic (particles of thrombus) or particles of the plaque. By having expandable loop 104 and porous filter 104 in a delivery configuration, embolic protection apparatus 100 is able to capture emboli, protecting the downstream lumens.

After the intervention is complete, the physician/user will retract the interventional device(s) leaving the embolic protection apparatus 100 in place. A retrieval catheter can then be advanced over wire 102 and filter 100 to capture filter 100 and enable its removal from the lumen.

In some embodiments shown, the wire 102 extends with the filter outside the porous filter 106. In these embodiments, the porous filter is attached to the expandable loop, and, in some embodiments, the distal end of the porous filter is attached to the wire. This attachment point may include a radiopaque marker. In other embodiments, wire 102 extends within the filter 104. In these embodiments, the porous filter 106 is attached to the expandable loop 104, and, in some embodiments, the distal end of the porous filter is attached to the wire, preferably at the point where the wire exits the porous filter. This attachment point may include a radiopaque marker. For all the embodiments shown herein, both of these two configurations are applicable.

In some embodiments, a single sized embolic protection apparatus will be used in lumens with a wide range of diameters. For example, an embolic protection apparatus with an expandable loop diameter of 12 mm can be used in lumens ranging from 5 to 10 mm.

The collapsible filters described herein may have a length of 2 cm to 7 cm. In some embodiments, the collapsible filter may have a length of 2.5 cm to 5 cm. In some embodiments the length of wire 102 that extends distally from the distal end of filter 106 can have a length of 10 cm 15 cm. In embodiments that have a segmented distal tip, each segment of the tip may have a length of 2 cm to 5 cm.

The device described herein may be used for two separate clinical indications. In many, but not all, cases both indications may exist. First, the device will serve as peripheral embolic protection device. Many endovascular procedures create unacceptable risk for peripheral embolizations, and many peripheral procedures are performed in presence of existing thrombus. The device will protect the patient from the risk of atheroemboli, and thromboemboli. Deployment of embolic protection basket distal to the lesion/thrombus will mitigate the risk of embolic complications during endovascular procedures. Its design and size can be tailored to peripheral arteries including the aorta, iliac, femoral, popliteal, common carotid, subclavian and brachiocephalic trunk. Its design and size can also be tailored to veins. Secondly, the devices design will allow the operator to easier cross chronic total occlusions (CTOs) of the above-mentioned arteries. In some cases, endovascular treatment of chronic total occlusions creates unacceptable risk of embolic complications, and the device described herein will allow the operator to treat CTOs in a safer, more intuitive, and expeditious manner. For example: presence of occluded peripheral graft with old thrombus creating the occlusion will always be associated with very high risk of embolic complications. The device described herein will significantly mitigate that risk.

The apparatuses of this disclosure are useful in a number of clinical situations. Lesions, including thrombotic occlusions, in the superficial femoral artery (SFA), common femoral artery, popliteal artery, iliac artery, iliac bypasses, or femoropopliteal (fem-pop) bypasses may be treated with the apparatus described here. Vessels that extend off the aortic arch such as the brachiocephalic artery, the right and left common carotid artery, brachiocephalic trunk, brachial branch, and the left subclavian artery can be treated with these devices. The apparatus described herein is also useful in the venous system and can be used to treat lesions in the iliac, femoral, popliteal, brachial, subclavian, axillary, innominate veins, and in the Inferior Vena Cava as well as Superior Vena Cava. Depending on the clinical requirements, either a radial, brachial, subclavian, pedal, proximal tibial, or femoral access can be used.

While many of the examples herein show and describe the devices and methods being used and performed in the vascular system, the devices and methods have applicability to non-vascular lumens.

The retrieval catheter, delivery catheter, and embolic protection apparatus will be constructed from materials that are known in the art. The delivery and retrieval catheters may have a multilayer or single layer construction. In a multilayer construction, the catheter could have a polymer inside layer, surrounded by a support structure such as a metal braid which in turn is surrounded by an outer polymer layer. Either catheter could have a flexibility that is consistent over the length of the catheter or could have increased flexibility at the distal end. Alternatively, the catheters could be made from a single or multi-stream extrusion, with or without an internal support structure. When one or both of the delivery and retrieval catheters have one or more marker bands, the marker bands can be formed of any radiopaque material and be in the form of a ring attached to either the internal or external surface, embedded in the internal or external surface so that they are flush with the surface, embedded within the wall structure of the catheter, or be a radiopaque agent mixed with the plastic of the catheter. One or both of the delivery and retrieval catheters can have a distal tip that is softer and/or more flexible that the body of the catheter. The embolic protection filter wire can be constructed of superelastic materials, nitinol, stainless steel, cobalt-chromium-nickel-molybdenum-iron alloy, or cobalt-chrome alloy, or a combination thereof. In embodiments where the basket and/or distal section(s) of the wire have a lesser diameter than the proximal section, the smaller diameter can be achieved by grinding or milling of the wire or by attaching a smaller diameter wire to the distal end of a larger diameter wire. The expandable loop can be constructed from superelastic materials, nitinol, stainless steel, cobalt-chromium-nickel-molybdenum-iron alloy, or cobalt-chrome alloy, or a combination thereof.

The porous filter 106 can be fabricated from a variety of different materials, such as, but not limited to, a woven or braided plastic or metallic mesh, a perforated polymer film, a shape memory material or mesh, combinations thereof, or other material that can be capable of capturing material within flowing blood, while allowing the blood to flow through the pores of the material. In some embodiments the porous filter comprises expanded polytetrafluoroethylene (ePTFE), polyurethane, polyolefin elastomers, polyamides, nylons, polyethers, polyamide block ethers (PEBAX), polyesters, and/or co-polyesters. In some embodiments the filter material has a thickness of 0.001 inches (25 microns) and the material has an 85 A Shore A Hardness. In some embodiments the filter material has a thickness of 0.0017 inches with an 80 Shore A Hardness. In some embodiments the porous filter can be woven or braided into a mesh and can be made from polyester, polyamide, polyurethane, nitinol, or stainless-steel filaments. The porous filter can have a variety of differently sized pores ranging from about 50 microns to about 200 microns, from about 60 microns to about 180 microns, or from about 75 microns to about 150 microns. For some applications, the pores can be sized up to 250 microns. The pores can have a variety of different configurations and can be circular, oval, polygonal, combinations thereof and the porous filter can include pores that are differently sized and configured. In practice, the pore size can vary as needed, so long as the pores are sized so that the pores do not compromise blood flow through the filter and collect emboli that can adversely affect downstream vessels. The porous filter can be coated with a hydrophilic coating, a heparinized coating, PTFE, silicone, combinations thereof, or other coatings. In some embodiments, the porous filter can be attached to the expandable loop by dip coating or by being wrapped around the loop and then sealed with heat or through an adhesive.

In some embodiments, the retrieval catheter will have a length of 120 to 140 cm with an outside diameter between 0.07 and 0.09 inches, preferably about 0.08 inches and with an inside diameter between 0.065 and 0.085 inches preferably about 0.07 inches. In some embodiments the delivery catheter will have a length of about 260 to 300 cm with an outside diameter between 0.06 and 0.08 inches preferably 0.06 inches and an inside diameter between 0.04 and 0.075 inches preferably 0.055 inches. In other embodiments, the delivery catheter will have a length of about 100 to 150 cm. In some embodiments, the catheter itself will have a length of 120 to 140 cm and the proximal wire will have a length of 120 to 160 cm. In some embodiments, the embolic protection device will have a length of 260 to 300 cm. In some embodiments, the wire will have a diameter of 0.035 inches. In embodiments where the basket and/or distal sections have a smaller diameter, they can have a diameter of 0.018 or 0.014 inches. In some embodiments where the delivery catheter is angled, the angled section 114 can be located 1 cm from the distal tip. In embodiments where the delivery catheter has one or more radiopaque markers, the distal marker can be located 1 cm from the distal end and the proximal band, if any, will be located 5 cm from the distal end. In some embodiments where the retrieval catheter is angled, the angled section 114 can be located 2 cm from the distal tip. In embodiments where the retrieval catheter has one or more radiopaque markers, the distal marker can be located 2 cm from the distal end. In embodiments where the distal section of the retrieval and/or delivery catheter are angled, they can be angled between 10 and 30 degrees away from the longitudinal axis of the catheter. In some embodiments, Nitinol wire is used to form the two legs and/or expandable frame. In some embodiments, radiopaque markers are provided on the wire of the two legs and/or expandable frame. In some embodiments, the wire of the two legs and/or expandable frame can be a cored wire, or a wire formed from two materials such as platinum and nitinol.

In some embodiments, a method for protecting a vessel comprises providing a distal protection device comprising a wire with a collapsible basket positioned near a distal end of the wire. The collapsible basket comprises an expandable loop with two legs, which are connected to the wire at a connection, and a porous filter attached at one end to the loop. In some embodiments, the two legs extend along the wire between the collapsible basket and the connection. In some embodiments, the distal protection device is positioned within a delivery catheter. The delivery catheter and the distal protection device are advanced through a lumen to a position downstream from a treatment area. The delivery catheter is withdrawn to allow the expandable loop to expand into contact with the interior of the lumen.

In some embodiments, a method of accessing a lesion within an occluded lumen comprises providing an embolic protection apparatus positioned within a delivery catheter. In some embodiments, the embolic protection apparatus comprises a wire, two legs, and an expandable loop with a porous filter. In some embodiments, the two legs connect the expandable loop to the wire at a connection, and the two legs extend along the wire between the collapsible basket and the connection. In some embodiments, a distal end of the wire extends past a distal end of the delivery catheter. In some embodiments, the distal end of the wire has a flexibility that allows the wire to loop back toward the proximal end of the delivery catheter. The delivery catheter and embolic protection apparatus are advanced through a body lumen to the site of a lesion by applying forward force to the delivery catheter or the wire, with the distal end of the wire looped over the distal end of the delivery catheter, until the distal end of the delivery catheter is distal of the lesion. The delivery catheter is retracted from over the collapsible basket and allowing the collapsible basket to assume an open configuration.

Although specific embodiments have been illustrated and described herein, those of ordinary skill in the art will appreciate that an arrangement calculated to achieve the same results can be substituted for the specific embodiments shown. For example, where the disclosure my show a system or a method with one example of a distal protection device, any distal protection device can be used including those disclosed herein. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An embolic protection apparatus comprising:
a wire with a collapsible basket positioned near a distal end of the wire; and the collapsible basket comprising an expandable loop with two legs, the two legs connected to the wire at a connection, and a porous filter attached at one end to the loop;
wherein the two legs extend along the wire between the collapsible basket and the connection.

2. The embolic protection apparatus of claim 1 wherein the expandable loop comprises a material which enables the expandable loop to expand to an open configuration from a delivery configuration.

3. The embolic protection apparatus of claim 1 wherein the two legs extend in a proximal direction from the expandable loop.

4. The embolic protection apparatus of claim 3 wherein a first of the two legs is connected to the wire on a first side of the wire and a second of the two legs is connected to the wire on a second side of the wire and wherein the first leg is connected to a section of the expandable loop that is positioned on the second side of the wire and the second leg is connected to a section of the expandable loop that is positioned on the first side of the wire.

5. The embolic protection apparatus of claim 1 where in a first leg of the two legs crosses the second leg of the two legs between the connection of the two legs to the wire and the expandable basket.

6. The embolic protection apparatus of claim 1 wherein the expandable loop comprises a preferential bend region.

7. The embolic protection apparatus of claim 6 wherein the preferential bend region is positioned approximately on the opposite side of the expandable loop from the two legs.

8. The embolic protection apparatus of claim 6 wherein the preferential bend region comprises a u-shape, a v-shape, or an omega-shaped region.

9. The embolic protection apparatus of claim 1 wherein the expandable loop, when expanded in an unconstrained fashion, has a non-round shape.

10. The embolic protection apparatus of claim 9 wherein the expandable loop, when positioned with the wire at the bottom of the loop, has a vertical diameter that is less than a horizontal diameter.

11. The embolic protection apparatus of claim 10 wherein the horizontal diameter is more than about 5% longer than the vertical diameter.

12. The embolic protection apparatus of claim 1 wherein the porous filter comprises holes and the holes are arranged in a staggered pattern.

13. An embolic protection apparatus comprising:
a wire with a collapsible basket positioned near a distal end of the wire;
the collapsible basket comprising an expandable loop connected to the wire, and a porous filter attached at one end to the loop; and
wherein the expandable loop has a non-round shape when in an unconstrained and expanded position.

14. The apparatus of claim 13 further comprising:
two legs, wherein an end of each of the two legs is connected to the loop and the other end of the two legs is connected to the wire.

15. The apparatus of claim 13 wherein the expandable loop comprises a preferential bend region.

16. The apparatus of claim 15 wherein the preferential bend region is positioned approximately on the opposite side of the expandable loop from the wire.

17. An embolic protection apparatus comprising:
a guide wire;
a support member comprising a first leg, a loop and a second leg, the first leg attached to the guide wire at a junction, the first leg extending distal to the junction, the second leg attached to the guide wire at the junction, the second leg extending distal to the junction, the loop extending distal to the first leg and the second leg, the loop defining an aperture, the guide wire extending through the aperture; and
a porous filter attached to the loop.

18. The embolic protection apparatus of claim 17, wherein the first leg is oriented to a first side of the guide wire and is attached to a portion of the loop oriented to a second side of the guide wire, and the second leg is oriented to the second side of the guide wire and is attached to a portion of the loop oriented to the first side of the guide wire.

19. The embolic protection apparatus of claim 17, comprising a first orientation and a second orientation, wherein the aperture is smaller in the second orientation than in the first orientation, and a distance between the first leg and the second leg is greater in the second orientation than in the first orientation.

20. The embolic protection apparatus of claim 17, the loop comprising a preferential bend region.
